(19) United States
(12) Patent Application Publication
(10) Pub. No.: US 2018/0193572 A1
(43) Pub. Date: Jul. 12, 2018

(54) SINGLE USE DELIVERY DEVICE

(71) Applicant: Marc Andrew Koska, East Sussex (GB)

(72) Inventor: Marc Andrew Koska, East Sussex (GB)

(21) Appl. No.: 15/741,011

(22) PCT Filed: Jul. 1, 2016
(86) PCT No.: PCT/IB2016/001027

§ 371 Date: Dec. 29, 2017

Related U.S. Application Data
(60) Provisional application No. 62/188,114, filed on Jul. 2, 2015.

(51) Int. Cl. A61M 5/50; A61M 5/28; A61M 5/46

(52) U.S. Cl. CPC A61M 5/50; A61M 2005/3128; A61M 5/46; A61M 5/282

(57) ABSTRACT

The invention is a single use delivery device capable of delivery of an agent (e.g., vaccine, drug, medicament, etc.) in a controlled manner and without requiring specialized skill in administering delivery of such agent. The delivery device is configured to be filled on-site and in the field with a dose of an agent, while remaining sterile and preventing the potential for contamination during the filling process. The delivery device is configured to be fitted with various types of administering components, including, but not limited to, needles, nozzles for producing spray or droplets, and the like. The delivery device is further configured to be rendered incapable of reuse followings its intended use of delivering the fluid agent to a patient, thereby preventing reuse of the device and reducing the risk of the spreading blood-borne diseases through reuse.

### CROSS REFERENCE TO RELATED APPLICATIONS

This application claims the benefit of and priority to U.S. Provisional Application No. 62/188,114, filed Jul. 2, 2015, the content of which is hereby incorporated by reference herein in its entirety.

### FIELD OF THE INVENTION

The present invention generally relates to delivery devices for delivering substances, such as medicaments, and, more particularly, to a single use delivery device that is rendered incapable of reuse following its intended use of delivering the fluid agent to a patient.

### BACKGROUND

Every year, millions of people become infected and die from a variety of diseases, some of which are treatable or entirely preventable. For example, many diseases may be prevented via immunization programs which include the administration of vaccines. Although vaccination has led to a dramatic decline in the number of cases of several infectious diseases, some of these diseases remain quite common. In many instances, large populations of the world, particularly in developing countries, suffer from the spread of vaccine-preventable diseases due to ineffective immunization programs, either because of poor implementation, lack of affordable vaccines, or inadequate devices for administering vaccines, or combinations thereof.

Some implementations of immunization programs generally include administration of vaccines via a typical reusable syringe. However, in many situations, particularly in developing countries, the administration of vaccines occur outside of a hospital and may be provided by a non-professional, such that injections are given to patients without carefully controlling access to syringes. The use of reusable syringes under those circumstances increases the risk of infection and spread of blood-borne diseases, particularly when syringes, which have been previously used and are no longer sterile, are used to administer subsequent injections. For example, the World Health Organization (WHO) estimates that blood-borne diseases, such as Hepatitis and human immunodeficiency virus (HIV), are being transmitted due to reuse of such syringes, resulting the death of more than one million people each year.

### SUMMARY

The present invention provides a single use delivery device that overcomes the drawbacks of current delivery devices and methods. In particular, the single use delivery device of the present invention is capable of delivering an agent (e.g., vaccine, drug, medicament, etc.) in a controlled manner and without requiring specialized skill in administering delivery of such agent. The delivery device is configured to be filled on-site and in the field with a dose of a fluid agent, while remaining sterile and preventing the potential for contamination during the filling process. Thus, because the delivery device itself is not prefilled, the delivery device of the present invention does not require the maintenance of a certain temperature (e.g., 2 to 8 degrees Celsius) during shipment or storage, thus cutting down on the overall costs. Rather than maintaining the delivery device at a constant temperature, as is the case with current devices, only the source containing the vaccine or drug (e.g., single supply provided in filling syringe) need by maintained at a constant temperature.

The delivery device is configured to allow delivery of the agent to the patient in a relatively simple manner, without requiring specialized training for administering the agent. In particular, the delivery device is designed such that a person administering the agent (e.g., administrator) need only position the device upon the administration site (e.g., shoulder, arm, chest, nose, ear, eye, etc.), and then fully compress a reservoir containing the dose of agent, thereby delivering the correct predefined dosage to the patient. Accordingly, the delivery device of the present invention does not require a trained, skilled healthcare profession for administration of vaccines or drugs. As such, the delivery device may be particularly useful in situations in which vaccines or drugs are being administered in non-healthcare related facilities (e.g., outside of clinics or hospitals) and given to large numbers of individuals over a short period of time by a non-professional. The delivery device further includes numerous safety features for preventing the potential for reuse, thereby reducing the risk of the spreading blood-borne diseases through reuse. For example, the delivery device is configured to be rendered incapable of reuse following its delivery of the agent to a patient.

In one aspect, the present invention provides a single use delivery device including an administration member for administering a fluid agent into a patient and a base member for providing the fluid agent into the administration member. The base member includes a proximal end having an inlet port configured to receive the fluid agent from a source and a distal end having an outlet port coupled to the administration member and configured to provide the fluid agent thereto. The base member includes a channel providing a fluid pathway from the inlet port to the outlet port and a one-way valve positioned within the fluid pathway of the channel, the one-way valve configured to limit fluid flow to an antegrade direction from the inlet port towards the outlet port.

The delivery device further includes a top member coupled to the base member. The top member includes a compressible reservoir member in fluid communication with the fluid pathway of the channel. The reservoir member has an interior volume configured to receive and store the fluid agent passing through the one-way valve. The reservoir member is further configured to expel the fluid agent into the fluid pathway and through the outlet port into the needle in response to a compression force applied thereto.

Accordingly, upon receiving a fluid agent from a source via the inlet port, the one-way valve is configured to only permit unidirectional flow of the fluid agent from the inlet port through the valve and towards the outlet port via the fluid pathway of the channel. Thus, when filling the delivery device with a fluid agent from a source (e.g. multi-dose source syringe, etc.), for example, a person need only couple the syringe to the inlet port and then fill the reservoir with the fluid agent by applying pressure to a plunger of the filler syringe. Due to the one-way valve, the fluid agent is only permitted to flow within the reservoir and prevented from flowing in a retrograde fashion out of the reservoir. Furthermore, the interior volume of the reservoir may be within a range considered to be a dose. Accordingly, rather than requiring a person to closely monitor the exact amount of fluid agent provided to the delivery device, they need only provide the fluid agent to the delivery device until the interior volume of the reservoir is completely filled (the interior volume is limited to the dosage amount for any given fluid agent).

In some embodiments, the administration member may include a needle for at least one of subcutaneous, intramuscular, intradermal, and intravenous injection of the fluid agent into the patient. In other embodiments, the administration member may include a nozzle configured to control administration of the fluid agent to the patient. The nozzle may include a spray nozzle, for example, configured to facilitate dispersion of the fluid agent into a spray. Accordingly, a delivery device fitted with a spray nozzle may be particularly useful in the administration of a fluid agent into the nasal passage, for example, or other parts of the body that benefit from a spray application (e.g., ear canal, other orifices). In other embodiments, the nozzle may be configured to facilitate formation of droplets of the fluid agent. Thus, a delivery device including a droplet nozzle may be useful in the administration of a fluid agent by way of droplets, such as administration to the eyes, topical administration, and the like.

In some embodiments, a seal member may cover the inlet port of the base member so as to prevent any contaminants from entering the inlet port and potentially contaminating the delivery device prior to filing the delivery device with the fluid agent. For example, a single use seal member composed of a relatively thin sheet of material (e.g., metal foil, plastic, etc.) may be hermetically sealed to the opening of the inlet port, thereby preventing contaminants (e.g., gases, fluids, dirt, debris, etc.) from entering the delivery device. The seal member is configured to rupture upon coupling of the filler syringe to the inlet port, thereby allowing a fluid to enter into the delivery device via the inlet port. Accordingly, the seal member provides a measure of security to ensure that the delivery device remains sterile until it is to be used. The seal member is generally applied to the delivery device during manufacture and/or assembly of the device. A plurality of empty delivery devices may then be shipped and stored at a desired location and will remain sterile, due, in part, to the seal member, thereby improving the process of storing such devices and the speed of assembly and use of such devices. This also may remove the requirement for individual blister packaging sleeves and allow for bulk packing. Bulk packing is a very big advantage in the market, reducing the individual unit production costs, handling, shipping and storage.

The delivery device may be configured to prevent unintentional needle sticks, or inadvertent contact with the administration member, thus reducing the potential for spreading blood-borne diseases. For example, in some embodiments, the base member further includes a protector member extending from distal end adjacent to the outlet port. The protector member is configured to move between a closed position, in which a tip of the administration member (e.g., needle, nozzle, etc.) is shielded, and an open position, in which the tip of the administration member exposed. Accordingly, needle protector member may be in a closed position while the delivery device is being shipped, stored, and handled (e.g., during filling of the delivery device). An administrator need only move the protector member to an open position to expose the administration member for delivering the fluid agent to a target site on a patient. Upon delivering the fluid agent, the administrator may then move the protector member to a closed position and discard the delivery device.

As previously described, delivery devices consistent with the present disclosure are not prefilled. Accordingly, rather than maintaining the individual delivery device at a constant temperature, as is the case with some current devices, only the source (e.g. filler syringe) containing the fluid agent need be maintained at a constant temperature. Additionally, because the reservoir member of the delivery device is configured to store and expel a micro dose of the fluid agent, the delivery device of the present invention allows for accurate dose-sparing. Accordingly, a plurality of empty delivery devices may be shipped and stored, at a reduced cost, and then filled directly on-site and on an as-needed basis, such that only a single filler syringe is required for hundreds of doses to be delivered at any given point. Additionally, because the delivery device is not prefilled, it may be sterilized at any point prior to being filled with the fluid agent, which further improves the bulk shipping and storage of such devices.

The base member and top member may be formed of medical grade materials. In some embodiments, the base member and top member may be formed from a thermoplastic polymer, for example. An advantage of the construction of the delivery device is that the base and top members may be produced separately from one another, wherein the base member may have a consistent production size and shape, while production of the top member may vary depending on the dosage amount. For example, certain vaccines require specific dosage amounts. Accordingly, a first production of top members can be produced so as to have a reservoir having an interior volume corresponding to a dosage amount recommended for a first vaccine (e.g., poliovirus vaccine) and a second production of top members can be produced so as to have a reservoir having an interior volume corresponding to dosage amount recommended for a second vaccine (e.g., Hepatitis). Accordingly, different dosage amounts can be easily produced (producing different top members) while still using a universal production of base members. The top member is then sealed to a base member to provide an assembled delivery device.

The delivery device is further configured to be rendered incapable of reuse following its delivery of the agent to a patient, thereby preventing reuse of the device and reducing the risk of the spreading blood-borne diseases through reuse. For example, in some embodiments, the reservoir member is configured to substantially collapse and reduce the interior volume upon substantial compression applied thereto. In particular, the top member may include an inelastic material such that the reservoir member is prevented from being reformed and the interior volume prevented from expanding subsequent to substantial compression. In some embodiments, the top member may further include a valve cover configured to substantially enclose the one-way valve within. Upon substantial compression applied to the valve cover, the valve cover is configured to substantially collapse upon the one-way valve and render the one-way valve inoperable, thereby blocking fluid flow from the inlet port to the reservoir member.

### BRIEF DESCRIPTION OF THE DRAWINGS

FIG. 1 is a perspective exploded view of a single use delivery device consistent with the present disclosure.

FIG. 2 is a top elevation view of the single use delivery device of FIG. 1 illustrating the base and top members in an assembled state.

FIG. 3 is side view of the single use delivery device of FIG. 1 illustrating the base and top members in an assembled state.

FIG. 4 is a perspective view of a single use delivery device consistent with the present disclosure having multiple inlet ports.

FIGS. 5 and 6 illustrate coupling of the single use delivery device of FIG. 1 to a source for providing a fluid agent to the single use delivery device.

FIGS. 7A-7C are side views of the single use delivery device of FIG. 1 illustrating different embodiments of needles to be used for intradermal, subcutaneous, and intramuscular delivery of a fluid agent, respectively.

FIG. 8 illustrates intradermal, subcutaneous, and intradermal delivery of a fluid agent with the single use delivery device of FIG. 1.

FIGS. 9A and 9B are perspective views of another embodiment of a needle protector in an open position, in which the penetrating tip of the needle is exposed, and a closed position, in which at least the penetrating tip of the needle is shielded and covered.

FIG. 10 is a side view of the single use delivery device of FIG. 1 including a spray nozzle for delivering a fluid agent.

FIG. 11 is a perspective of the single use delivery device of FIG. 1 including another embodiment of a spray nozzle for delivering a fluid agent, particularly within the nasal cavity of a patient.

FIG. 12 is a side view of the single use delivery device of FIG. 1 including a droplet nozzle for delivering a fluid agent.

FIG. 13 is a perspective view of the single use delivery device of FIG. 1 including another embodiment of a droplet nozzle for delivering a fluid agent to a patient's eyes or via oral route of administration.

FIG. 14 is a perspective view of the single use delivery device of FIG. 1 including another embodiment of an administration member for delivering a fluid agent within a patient's ear canal.

FIG. 15 is a perspective view of the single use delivery device of FIG. 1 including another embodiment of an administration member for delivering a fluid agent, particularly useful for topical application to a wound or other site.

### DETAILED DESCRIPTION

The present invention provides a single use delivery device that is capable of delivery an agent (e.g., vaccine, drug, medicament, etc.) in a controlled manner and without requiring specialized skill in administering delivery of such agent. The delivery device is configured to be fitted with various types of administering components, including, but not limited to, needles, nozzles for producing spray or droplets, and the like. The delivery device is configured to be rendered incapable of reuse following its intended use of delivering the fluid agent to a patient, thereby preventing reuse of the device and reducing the risk of the spreading blood-borne diseases through reuse.

By way of overview, the present invention provides a single use delivery device including an administration member for administering a fluid agent into a patient and a base member for providing the fluid agent into the administration member. The base member includes a proximal end having an inlet port configured to receive the fluid agent from a source and a distal end having an outlet port coupled to the administration member and configured to provide the fluid agent thereto. The base member includes a channel providing a fluid pathway from the inlet port to the outlet port and a one-way valve positioned within the fluid pathway of the channel, the one-way valve configured to limit fluid flow to an antegrade direction from the inlet port towards the outlet port.

The delivery device further includes a top member coupled to the base member. The top member includes a compressible reservoir member in fluid communication with the fluid pathway of the channel. The reservoir member has an interior volume configured to receive and store the fluid agent passing through the one-way valve. The reservoir member is further configured to expel the fluid agent into the fluid pathway and through the outlet port into the needle in response to a compression force applied thereto.

In some embodiments, the administration member may include a needle for at least one of subcutaneous, intramuscular, intradermal, and intravenous injection of the fluid agent into the patient. In other embodiments, the administration member may include a nozzle configured to control administration of the fluid agent to the patient. The nozzle may include a spray nozzle, for example, configured to facilitate dispersion of the fluid agent into a spray. Accordingly, a delivery device fitted with a spray nozzle may be particularly useful in the administration of a fluid agent into the nasal passage, for example, or other parts of the body that benefit from a spray application (e.g., ear canal, other orifices). In other embodiments, the nozzle may be configured to facilitate formation of droplets of the fluid agent. Thus, a delivery device including a droplet nozzle may be useful in the administration of a fluid agent by way of droplets, such as administration to the eyes, topical administration, and the like.

The delivery device is configured to allow delivery of the agent to the patient in a relatively simple manner, without requiring specialized training for injecting a needle portion intradermally. In particular, the delivery device is designed such that a person administering the agent (e.g., administrator) need only position the device upon the administration site (e.g., shoulder, arm, chest, nose, ear, eye, etc.), and then fully compress a reservoir containing the dose of agent, thereby delivering the correct predefined dosage to the patient. Accordingly, the delivery device of the present invention does not require a trained, skilled healthcare profession for administration of vaccines or drugs. As such, the delivery device may be particularly useful in situations in which vaccines or drugs are being administered in non-healthcare related facilities (e.g., outside of clinics or hospitals) and given to large numbers of individuals over a short period of time by a non-professional. The delivery device further includes numerous safety features for preventing the potential for reuse, thereby reducing the risk of the spreading blood-borne diseases through reuse. For example, the delivery device is configured to be rendered incapable of reuse following its delivery of the agent to a patient.

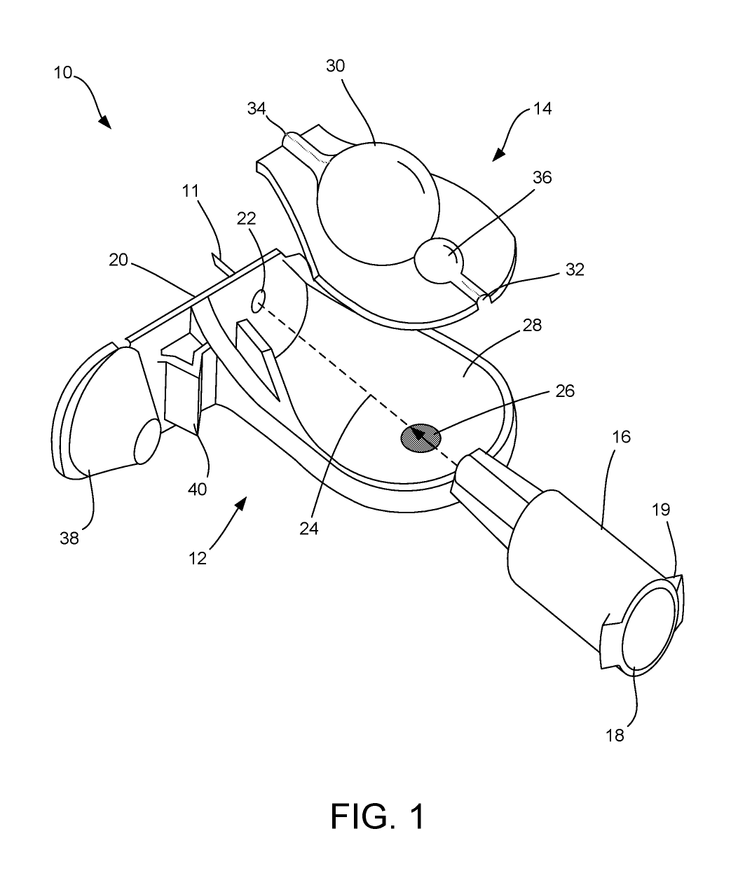

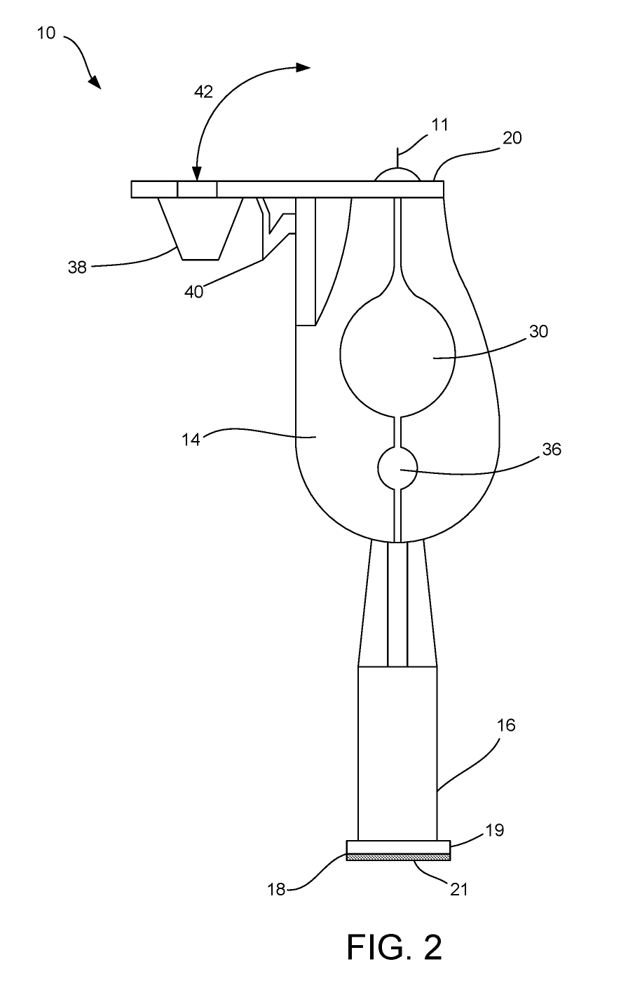

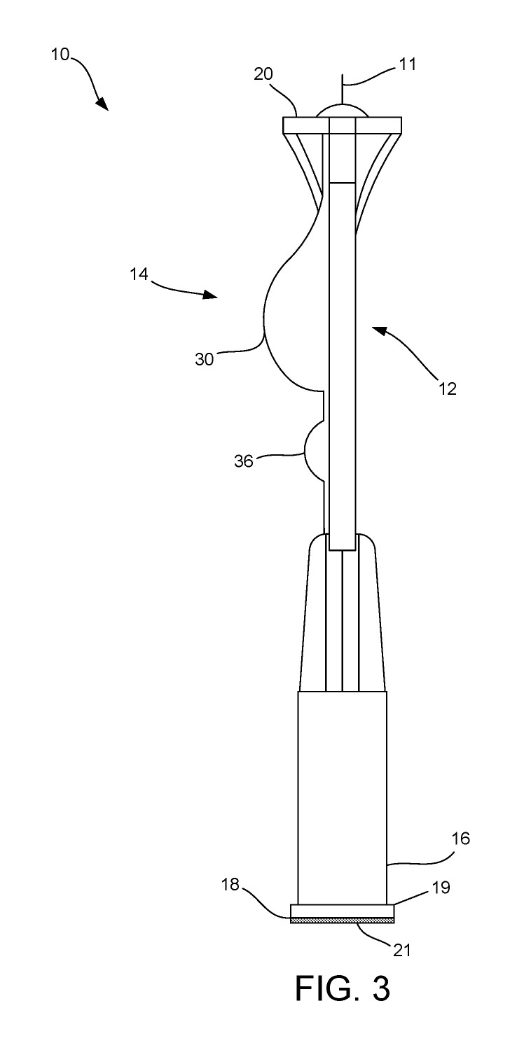

FIG. 1 is a perspective exploded view of a single use delivery device 10 consistent with the present disclosure. FIGS. 2 and 3 are top and side elevation views of the single use delivery device 10 of FIG. 1 in an assembled state. As shown, the single use delivery device 10 may include a needle 11 having a tip configured for penetrating a target site and injecting a fluid agent into the target site. As will be described in greater detail herein, the needle may include a micro needle configured to penetrate a patient's skin down to a depth of the dermis and deliver a dosage of fluid agent thereto. In other embodiments, however, the needle 11 may be sized for other injection types (e.g., intravenous, subcutaneous, intradermal, etc.). In some embodiments, the single use delivery device 10 of the present disclosure is not limited solely to the administration of a fluid agent via injection, and thus may be fitted with other means of delivering a fluid agent (e.g., nozzle tip, spray tip, droplet tip, etc.) in lieu of a needle.

The device 10 further includes a base member 12 and a top member 14 coupled thereto, wherein the combined base and top members 12, 14 are configured to provide the fluid agent into the needle for subsequent injection. As generally understood, the fluid agent may include any type of agent to be injected into a patient (e.g., mammal, either human or non-human) and capable of producing an effect. Accordingly, the agent may include, but is not limited to, a vaccine, a drug, a therapeutic agent, a medicament, or the like.

The base member 12 includes a proximal end 16 having an inlet port 18 configured to receive fluid agent from a source and a distal end 20 having an outlet port 22 coupled to the needle 11 and configured to provide the fluid agent thereto. As described in greater detail herein, the source of the fluid agent may include a filling syringe, for example, configured to be releasably coupled to the inlet port 18 of the base member 16. As shown, the inlet port 18 may include a Luer-type connection 19, such as a Luer-Lok fitting, configured to releasably engage a corresponding Luer-type connection on a hub of the syringe, thereby providing a fluid connection between the syringe and the inlet port 18 of the base member 12. It should be noted that the inlet port 18 need not be limited to an ISO standard (e.g. ISO 594) luer fitting. In other embodiments, the inlet port 18 may include non-standard connection fittings to be coupled with non-standard connection fitting of a source or adapter, for example. Accordingly, by providing a specialty connection fitting, only approved sources (e.g., multi-dose dispensing devices) can be used with the delivery devices of the present disclosure, thereby adding one more layer of security.

As shown, a seal member 21 may cover the inlet port 18 so as to prevent any contaminants from entering the inlet port 18 and potentially contaminating the delivery device 10 prior to filing the delivery device 10 with the fluid agent. For example, a single use seal member 21 may be composed of a relatively thin sheet of material (e.g., metal foil, plastic, etc.) may be hermetically sealed to the opening of the inlet port 18, thereby preventing contaminants (e.g., gases, fluids, dirt, debris, etc.) from entering the delivery device 10. The seal member 21 may be coupled to the inlet port 18 by any known sealing techniques (e.g., heat, vibration, or adhesive process). The seal member 21 is configured to be durable in the sense that it provides a sufficient seal with the inlet port 18 and prevent contaminants from entering into the device 10 via the inlet port 18 while also being configured to be pliable and rupture upon coupling of the inlet port 18 to a source (e.g., hub of filler syringe), thereby allowing a fluid to enter into the delivery device 10 via the inlet port 18. Accordingly, the seal member 21 provides a measure of security to ensure that the delivery device 10 remains sterile until it is to be used.

The base member 12 may further include a channel 24 formed within a portion thereof and providing a fluid pathway from the inlet port 18 to the outlet port 22. Accordingly, upon receipt of fluid agent from a source, via the inlet port 18, the fluid agent may flow within the pathway provided by the channel 24. The base member 12 further includes a one-way valve 26 positioned within the fluid pathway of the channel 24. The one-way valve 26 is configured to permit antegrade flow of fluid from the inlet port 18 to the outlet port 22, while preventing retrograde flow (e.g., backflow) of fluid from the outlet port 22 through the valve 26 and through the inlet port 18. For example, the one-way valve 26 may include an open inlet end and an adjustable outlet end configured to move between a normally closed position and an open position. The one-way valve 26 is positioned such that the open inlet end is configured to receive fluid from the inlet port 18, and, upon sufficient application of fluid pressure in a direction away from the inlet port 18 and towards the outlet port 22 (e.g., depressing plunger of filling syringe to fill device 10 with fluid agent) the outlet end of the valve 26 moves from the normally closed position to an open position to allow fluid to flow therethrough in a direction towards the outlet port 22, as indicated by the directional arrow. However, when in a closed position, the outlet provides a substantially leak-proof and/or airtight seal so as to prevent any fluid from entering the valve 26 from the outlet end. Furthermore, the valve 26 is configured such that any application of fluid pressure in a direction away from the outlet port 22 and towards the outlet end of the valve 26, the outlet end remains closed, thereby preventing any fluid from flowing through the valve 26 in a retrograde direction from the outlet port 22 towards the inlet port 18. As generally understood, the one-way valve 26 may include any type of valve configured to permit fluid to flow only in a single direction. The one-way valve 26 may include any type of valve having medical grade material and configured to be used with the flow of fluids. For example, the one-way valve 26 may include a Reed valve or a Heimlich valve.

The top member 14 may be formed separately from the base member 12, which provides advantages, as previously described herein. Accordingly, the top member 14 may be coupled to a portion of the base member 12 along a mounting section 28. For example, the mounting section 28 generally includes a large portion of the base member 12 and includes at least a portion of the channel 24 and the one-way valve 26, such that, upon coupling the top member 14 to the mounting section 28 of the base member 12, the top member substantially encloses the channel 24 and the one-way valve 26.

The top member 14 includes a compressible reservoir member 30 and a compressible valve cover 36, such that, upon coupling the top member 14 to the base member 12, the reservoir member 30 is in fluid communication with the fluid pathway of the channel 24 and the valve cover 36 substantially encloses the one-way valve 26. The top member 14 may further include an inlet 32 and an outlet 34 and defining a fluid pathway extending there between and in fluid communication with the reservoir member 30 and valve cover 36. Accordingly, once coupled to the base member 12, the inlet 34 and outlet 34 and the pathway extending there between may substantially correspond to the fluid pathway of the channel 24, thereby cooperating with one another to form a combined single channel pathway from the inlet port 18 to the outlet port 22.

The top member 14 may be coupled to the base member 12 by any known means so as to create a hermetic seal. For example, the base and top members 12, 14 may be sealed with one another via any known adhesives, cements, ultrasonic welding, or thermoplastic bonding techniques. The base and top members 12, 14 are composed of a medical grade material. In some embodiments, the base member 12, the top member 14, or both, may be composed of a thermoplastic polymer, including, but not limited to, polypropylene, polyethylene, polybenzimidazole, acrylonitrile butadiene styrene (ABS) polystyrene, polyvinyl chloride, PVC, or the like.

The reservoir member 30 includes an interior volume configured to receive and store a fluid agent passing through the one-way valve 26. Upon applying a compression force to the reservoir member 30, the fluid agent is expelled into the fluid pathway of the channel 24 and through the outlet port 22 into the needle 11. Accordingly, the method of delivering the fluid agent into a patient is a relatively simple and straightforward process which simply requires an administrator to apply sufficient pressure to the filled reservoir member 30 so as to deform the reservoir, resulting in expulsion of the stored fluid agent from the interior volume. Due to the one-way valve 26, the fluid agent is force to flow in a direction towards the outlet port 22 and out of the needle 11.

The base member 12 further includes a needle protector member 38 extending from the distal end 20 and adjacent to the outlet port 22. The needle protector member 38 may be coupled to the distal end 20 by way of any known means. In the illustrated embodiment, the needle protector member 38 is coupled to the distal end 20 by way of a living hinge 40, for example. Accordingly, the needle protector member 38 is configured to move between a closed position and an open position, as indicated by arrow 42. When in a closed position, the needle protector member 38 is configured to substantially enclose the penetrating tip of the needle 11, thereby shielding one from inadvertent needle sticks. When in an open position, as shown, the penetrating tip of the needle 11 is exposed and ready for intradermal injection on a target site of a patient. Accordingly, the needle protector member 38 may be in a closed position while the delivery device 10 is being shipped, stored, and handled (e.g., during filling of the delivery device 10). An administrator need only move the needle protector member 38 to an open position to expose the needle 11 for delivering the fluid agent to a target site on a patient. Upon delivering the fluid agent, the administrator may then move the needle protector member 38 to a closed position and discard the delivery device 10, so as to prevent unintentional needle sticks.

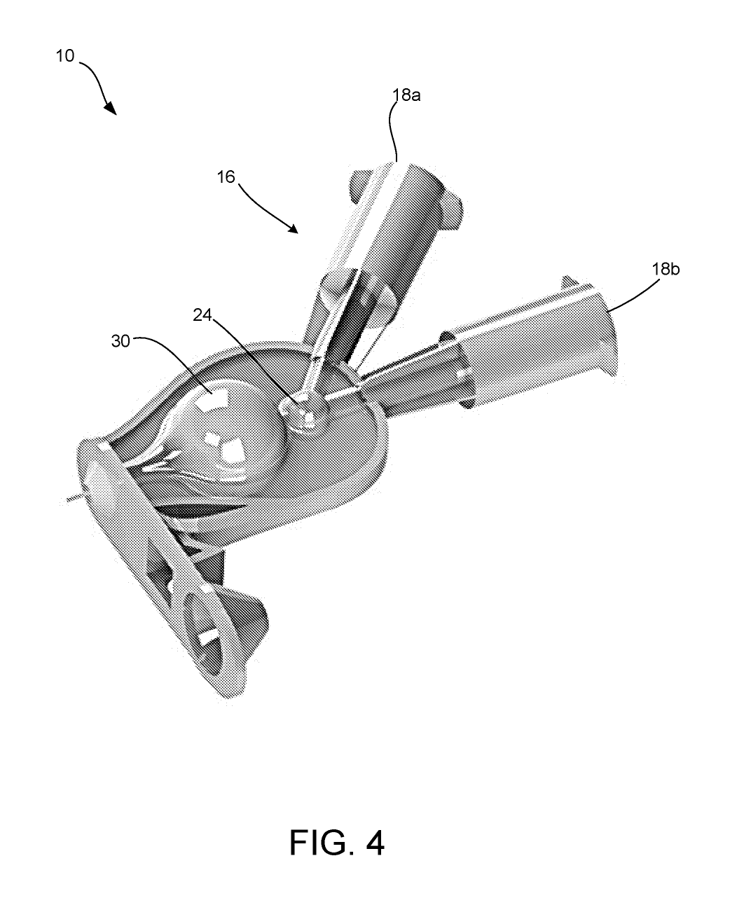

FIG. 4 is a perspective view of a single use delivery device 10 having multiple inlet ports. As shown, the proximal end 16 of the device 10 may include at least two inlet ports 18a, 18b, each configured to receive a separate fluid agent from a separate source, or, in some instances, the same fluid agent from the same source. As shown, each of the inlet ports 18a, 18b includes a separate fluid pathway coupled to the one-way valve 26. Accordingly, the one-way valve 26 is configured to permit antegrade flow of first and second fluids from inlet ports 18a, 18b, respectively, in a direction towards the outlet port 22 and into the reservoir member 30, while preventing retrograde flow.

The multiple inlet ports 18a, 18b allow for two separate fluids to be loaded into the device 10 and subsequently mixed within the reservoir member 30. This may be particularly useful in situations in which a therapeutic agent or medicament is in concentrated form and must be diluted prior to administration to a patient. For example, inlet port 18a may receive a fluid concentrate and inlet port 18b may receive a diluent fluid (e.g., saline), wherein the fluid concentrate may be mixed with the diluent fluid within the reservoir member 30. Accordingly, certain fluid agents or medicaments, such as certain vaccines, may be shipped, or otherwise stored, in a concentrated form and then diluted on-site when loading devices 10. The inclusion of multiple ports 18a, 18b thus allows for administration of a multivalent dose, which can be loaded and mixed at the point of use.

The inlet ports 18a, 18b may each include a connection fitting for coupling the inlet port 18a, 18b to a source (e.g., filler syringe, other multi-dose dispensing device, etc.) for dispensing a specific fluid into the respective inlet port 18a, 18b, wherein the connection fitting may be associated with a specific fluid. For example, at least one of the inlet ports may include a standard Luer-type connection, such as a Luer-Lok fitting, associated with a diluent fluid, while the other inlet port may include a non-standard connection fitting associated with a fluid concentrate. For example, inlet port 18a may include a Luer-Lok fitting configured to releasably engage a corresponding Luer-type connection on a hub of a filler syringe to thereby provide a fluid connection between the syringe and the inlet port 18a. The standard Luer-Lok fitting may be associated with a filler syringe for dispensing saline, thus providing visual indication to a user that inlet port 18a is to be coupled to a saline source and receive saline fluid within. The inlet port 18b may include a non-standard connection fitting (e.g., a non-ISO standard ISO 594 fitting) which may have specific dimensions, geometry, and the like and configured to fit with associated connection fitting of a source containing the concentrated vaccine. Accordingly, the non-standard connection fitting allows for only a corresponding source to be coupled thereto and further provide visual indication to a user that the concentrated vaccine is to be coupled to the inlet port 18b.

The delivery device is configured to allow delivery of the agent to the patient in a relatively simple manner, without requiring specialized training for injecting a needle portion intradermally. In particular, the delivery device is designed such that it may be filled on-site and in the field with a microdose of an agent, while remaining sterile and preventing the potential for contamination during the filling process.

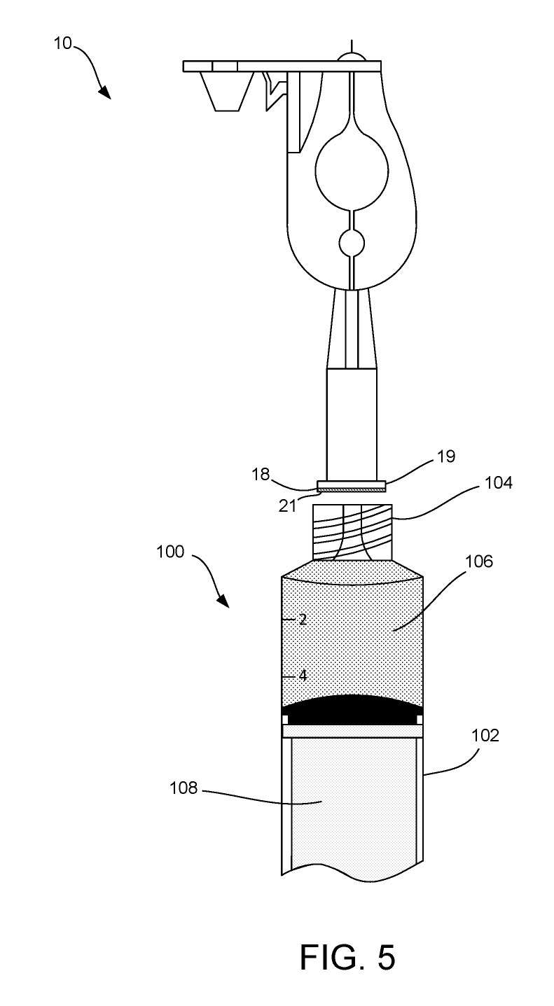

For example, FIGS. 5 and 6 illustrate coupling of the single use delivery device 10 to a multi-dose source for dispensing a fluid agent into the delivery device 10. In the illustrated embodiment, the source may include a filler syringe 100, for example. The filler syringe 100 may be embodied as a conventional syringe. Accordingly, the filler syringe 100 includes a barrel 102 having a distal hub 104 configured to be releasably coupled to the inlet port 18 of the base member 12 of the delivery device 10. For example, the inlet port 18 may include a Luer-type connection 19, such as a Luer-Lok fitting, configured to releasably engage a corresponding Luer-type connection on the hub 104 of the syringe 100, thereby providing a fluid connection between the interior volume of the barrel 102 of the syringe 100 and the inlet port 18 and subsequent fluid pathway formed by the channel 24 of the base member 12.

In order to fill the delivery device 10, specifically the reservoir member 30, with a fluid agent 106 contained with the syringe 100, a person need only couple the hub 104 with the inlet port 18. As shown in FIG. 5, the seal member 21 is intact and covering the inlet port 18 so as to prevent any contaminants from entering the inlet port 18 and potentially contaminating the delivery device 10 prior to filing the delivery device 10 with the fluid agent. Upon inserting the hub 104 into engagement with the inlet port 18, the hub 104 is configured to pierce the seal member 21, upon which the seal member 21 ruptures and tears, as indicated by arrow 43, thereby breaking the hermetic seal and allowing fluid to be providing from the syringe 100 into the device 10 through the inlet port 18. For example, upon rotating either the syringe 100 or device 10, as indicated by arrow 44, the hub 104 and inlet port 18 may contact and come into threaded engagement. A person may then fill the reservoir 40 with the fluid agent 106 by applying pressure to a plunger 108 of the filler syringe 100, as indicated by arrow 46. Due to the one-way valve 26, the fluid agent 106 is only permitted to flow in a direction towards the reservoir 30 and prevented from flowing in a retrograde fashion out of the reservoir 30. Furthermore, the interior volume of the reservoir 30 may be within a range considered to be a micro dose, such as 0.05 ml to 1.0 ml. Accordingly, in some embodiments, the delivery device 10 does not require exact measurements when filling the reservoir 30. Instead, a person need only completely fill the reservoir, which includes the correct dosage, and, once completely filled, the correct dosage has been reached and the buildup of pressure will prevent the plunger 108 of the syringe 100 from advancing further. Accordingly, the device 10 allows consistent filling and dosing of the fluid agent 106 from device to device (e.g., filling up tens of hundreds of devices 10 at any one time). Accordingly, when in the field or directly on-site, a person may use a single filling syringe 100 to fill a plurality of empty delivery devices 10 in a consistent manner. The filling syringe 100 essentially acts as a means of storing and dispensing aliquots of the fluid agent.

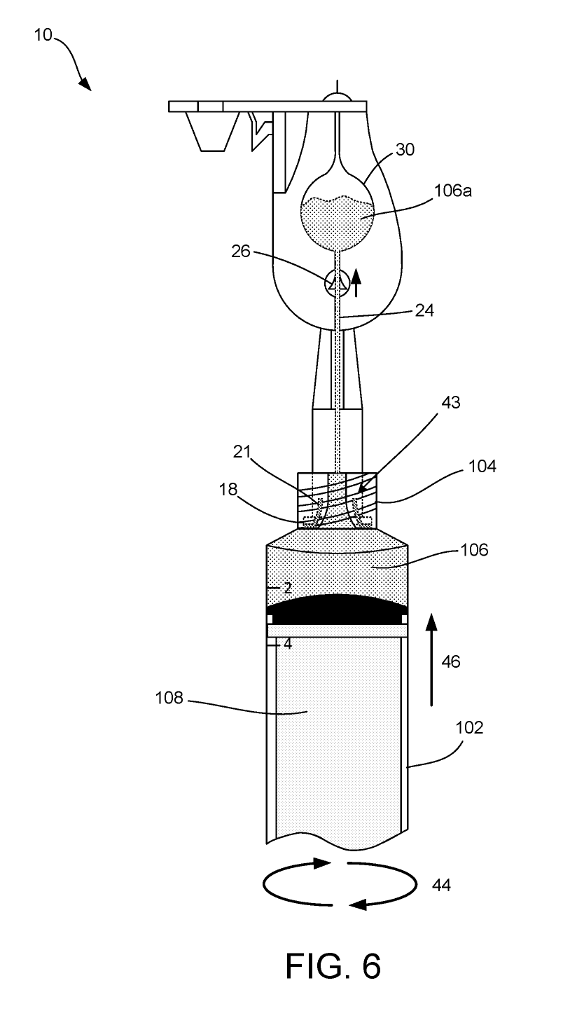

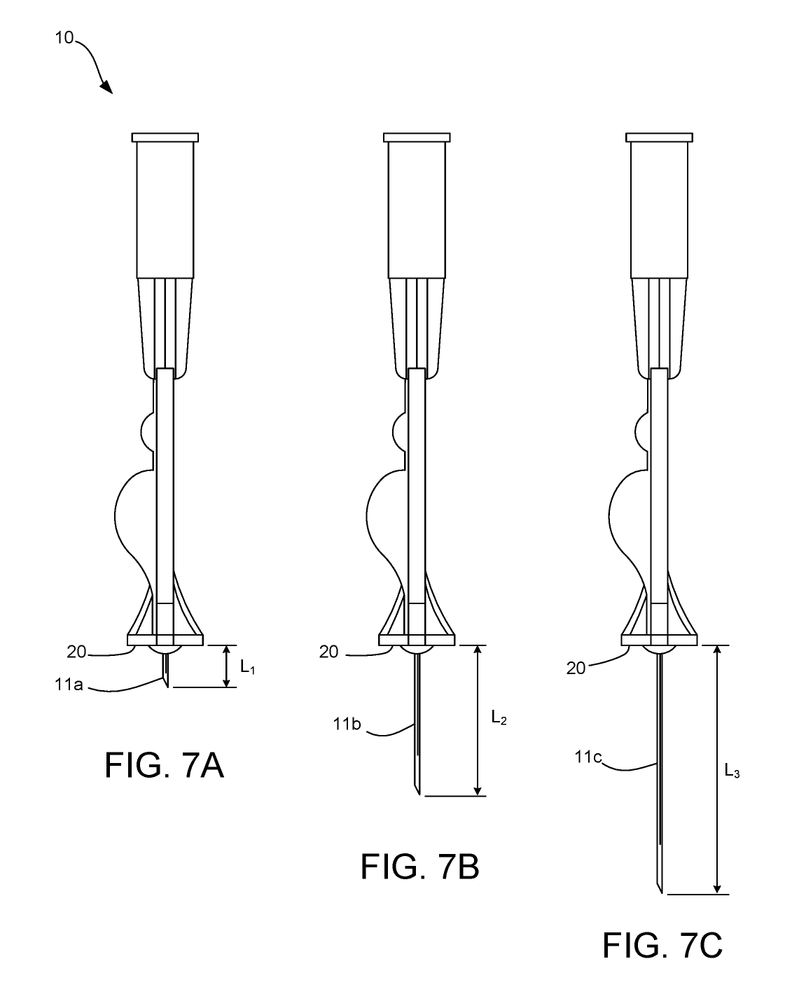

Once filled, the delivery device 10 is designed such that a person administering the agent (e.g., administrator) may easily administer a dose of the fluid agent as intended. For example, FIGS. 7A-7C are side views of the single use delivery device 10 illustrating different embodiments of needles to be used for intradermal, subcutaneous, and intramuscular delivery of a fluid agent, respectively. FIG. 8 illustrates intradermal, subcutaneous, and intradermal delivery of a fluid agent with the single use delivery device 10.

The delivery device 10 is configured to allow delivery of the agent to the patient in a relatively simple manner, without requiring specialized training for injecting a needle portion intradermally. In particular, the delivery device is designed such that a person administering the agent (e.g., administrator) need only press the delivery device against the administration site (e.g., shoulder, arm, chest, etc.), in which the device is configured such that needle penetration is limited to the correct length and orientation within the administration site. As shown, the delivery device 10 may be removed from the filler syringe 100 and used to administer the fluid agent as a standalone device. However, it should be noted that the delivery device 10 may remain coupled to the filler syringe 100 during administration of the fluid agent, such that an administrator may use the filler syringe 100 as a handle or means of stabilizing the delivery device 10 during delivery of the fluid agent to a patient.

As shown in FIG. 7A, the needle 11a is positioned substantially perpendicular relative to a plane along which the distal end 20 of the base member 12 lies, such that the needle 11a is configured to be inserted into a patient's skin at a substantially perpendicular angle. This is a much more straightforward process for intradermal delivery of an agent, particularly when compared to the Mantoux procedure. Furthermore, the distal end is configured to contact the patient's skin during penetration of the needle 11a, thereby indicating adequate depth of penetrating for intradermal injection of the fluid agent. For example, the needle 11a may be a micro-needle having a length L₁ (measured from the distal end 20) in the range of 0.5 mm to 4 mm.

Other needles may be used with devices 10 of the present disclosure. For example, as shown in FIG. 7B, the device 10 may include a needle 11b specifically designed for subcutaneous delivery of an agent. For example, the needle 11b may have a length L₂ (measured from the distal end 20) in the range of 8 mm to 15 mm. As shown in FIG. 7C, the device 10 may include a needle 11c specifically designed for intramuscular delivery of an agent, such that the 11c has a length L₃ (measured from the distal end 20) in the range of 18 mm to 30 mm.

Accordingly, as shown in FIG. 8, upon an administrator applying pressure in a direction towards the target site, as indicated by arrow 62, the needle 11a is configured to penetrate the epidermis and dermis layers of skin. Needle 11b is configured to penetrate the epidermis, dermis and subcutaneous layers. Needle 11c is configured to penetrate the epidermis, dermis, subcutaneous, and muscle layers. Upon sufficient contact between the distal end of the base member 12 and the outer layer of skin, as indicated by arrow 64, the needles 11a, 11b, 11c have achieved adequate penetration into the dermis for injection of the fluid agent into the appropriate layer. For example, upon the needle 11a reaching the adequate depth into the dermis, the administrator may then compress the reservoir member 30 containing the dosage of fluid agent so as to deliver the fluid agent into the dermis. For example, the reservoir member 30 is configured to substantially collapse and reduce the interior volume upon substantial compression applied thereto, as indicated by arrow 66. An administrator need only fully compress the reservoir member 30 so as to expel to required dosage. Upon compression of the reservoir member 30, the fluid agent is expelled into the fluid pathway of the channel 24 and out of the outlet port 22 and out of the needle 11, resulting in delivery of the fluid agent into the dermis, as indicated by arrow 68.

In some embodiments, the reservoir member 30 is shaped or sized such that, upon compression applied thereto, the reservoir member 30 is prevented from being reformed and the interior volume is prevented from expanding subsequent to substantial compression. Additionally, or alternatively, the valve cover 36 may be shaped or sized such that, upon compression applied thereto, the valve cover 36 is configured to substantially collapse upon the one-way valve 26 and render the one-way valve 26 inoperable, thereby blocking fluid flow into or out of the one-way valve 26. Accordingly, the delivery device 10 configured to be rendered incapable of reuse following its delivery of the agent to a patient, thereby preventing reuse of the device and reducing the risk of the spreading blood-borne diseases through reuse.

Accordingly, the delivery device 10 of the present invention does not require a trained, skilled healthcare profession for administration of vaccines or drugs. As such, the delivery device may be particularly useful in situations in which vaccines or drugs are being administered in non-healthcare related facilities (e.g., outside of clinics or hospitals) and given to large numbers of individuals over a short period of time by a non-professional.

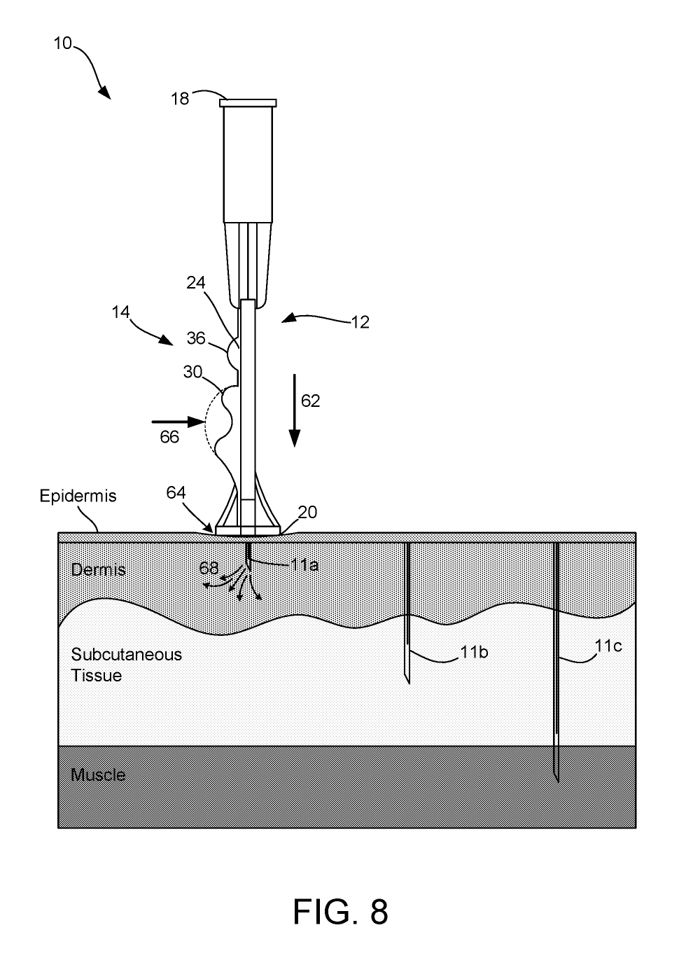

It should further be noted that, in order to compensate for the variety of different lengths of needles 7a-7c, the device 10 may further include an alternative embodiment of a needle protector. FIGS. 9A and 9B are perspective views of a needle protector member 70 in an open position, in which the penetrating tip of the needle 11 is exposed, and a closed position, in which at least the penetrating tip of the needle 11 is shielded and covered by the needle protector member 70. Similar to needle protector member 38 previously described herein, needle protector member 70 generally extends from the distal end 20 of the device 10 and is adjacent to the outlet port. The needle protector member 70 may be coupled to the distal end 20 by way of any known means. In the illustrated embodiment, the needle protector member 70 is coupled to the distal end 20 by way of a living hinge, for example. Accordingly, the needle protector member 70 is configured to move between a closed position and an open position. The needle protector member 70 is shaped and/or sized so as to accommodate needles of a specific length (e.g., needles having a length between 0.5 and 30 mm or longer). For example, when in a closed position, as shown in FIG. 9B, the needle protector member 70 is configured to substantially enclose at least the penetrating tip of a needle 11, wherein the needle may have a length between 4 mm and 30 mm or longer, such that the needle protector member 38 would be inadequate and would not accommodate a needle of such length. When in an open position, as shown in FIG. 9A, the penetrating tip of the needle 11 is exposed and ready for intradermal injection on a target site of a patient.

As generally understood, a therapeutic agent, medicament, fluid agent, drug, or the like, can be administered to a patient in a variety of different ways, or different routes of medication administration. A route of administration is the path by which therapeutic agent, medicament, fluid agent, drug is taken into the body. Common routes of administration include, but are not limited to, oral, intravenous, intradermal, intramuscular, subcutaneous, transdermal, epicutaneous or topical, nasal and transmucosal, intraocular or intravitreal (through the eye), and others.

It should be noted that the single use delivery device 10 of the present disclosure is not limited solely to the administration of a fluid agent via an injection needle (e.g., intravenous, intramuscular, intradermal, or subcutaneous route of administration). For example, in other embodiments, a delivery device 10 consistent with the present disclosure may be fitted with an administration member configured to deliver a fluid agent via a different route of administration in lieu of the use of a needle.

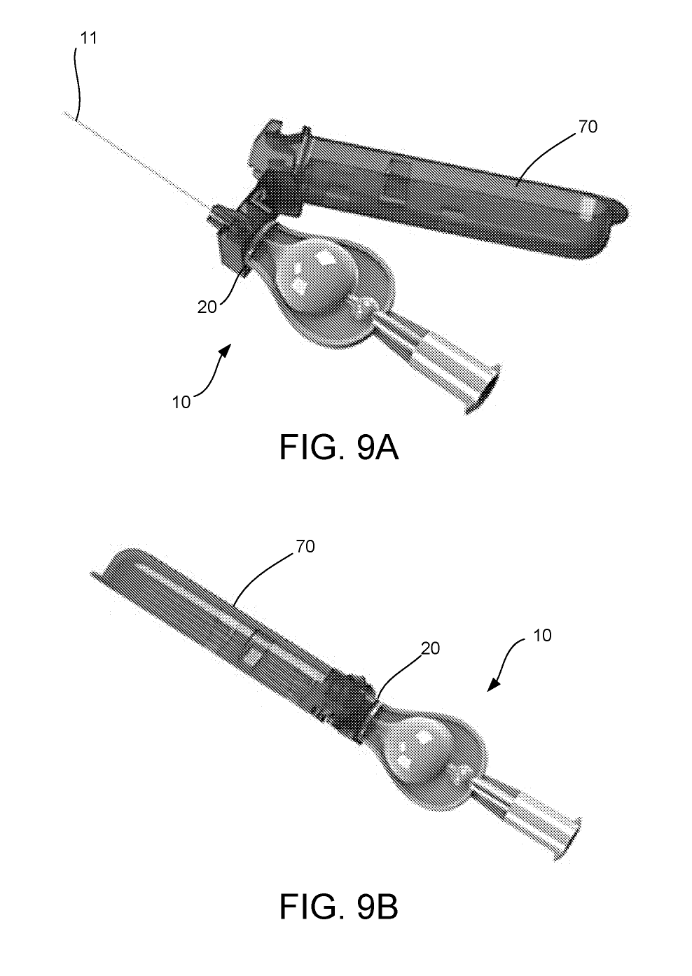

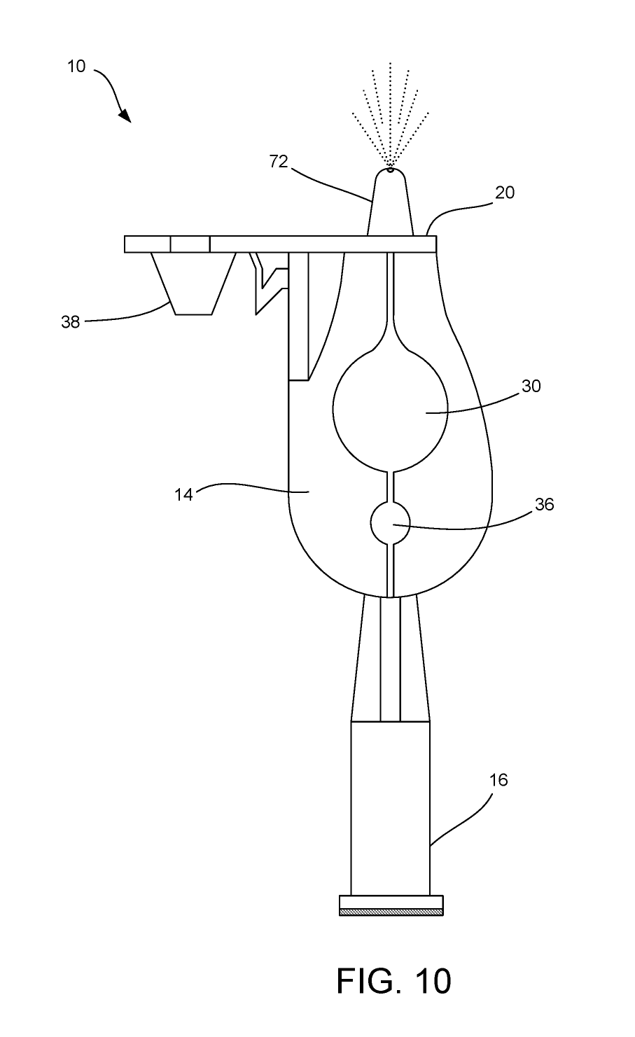

FIG. 10 is a side view of the single use delivery device 10 including an administration member 72 for delivering a fluid agent. The administration member 72 is generally fitted to the outlet port 22 or integrally formed with the device 10. The administration member 72 is configured to facilitate dispersion of the fluid agent within the reservoir member 30 into a spray upon compression of the reservoir member. Thus, the administration member 72 generally resembles a spray nozzle. FIG. 11 is a perspective of the single use delivery device 10 including another embodiment of a spray nozzle 74 for delivering a fluid agent. Accordingly, the delivery device 10 fitted with a spray nozzle 72, 74 may be particularly useful in the administration of a fluid agent into the nasal passage, for example, or other parts of the body that benefit from a spray application (e.g., ear canal, other orifices).

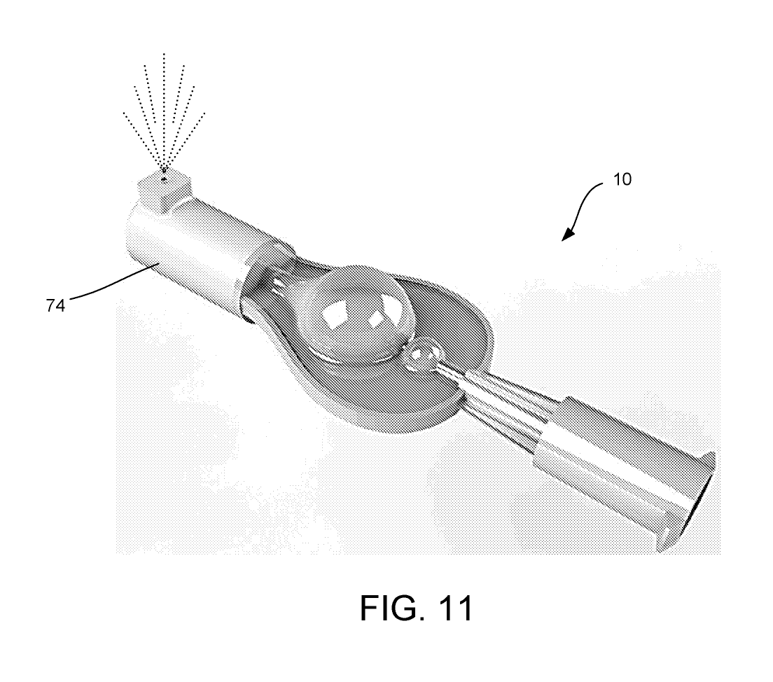

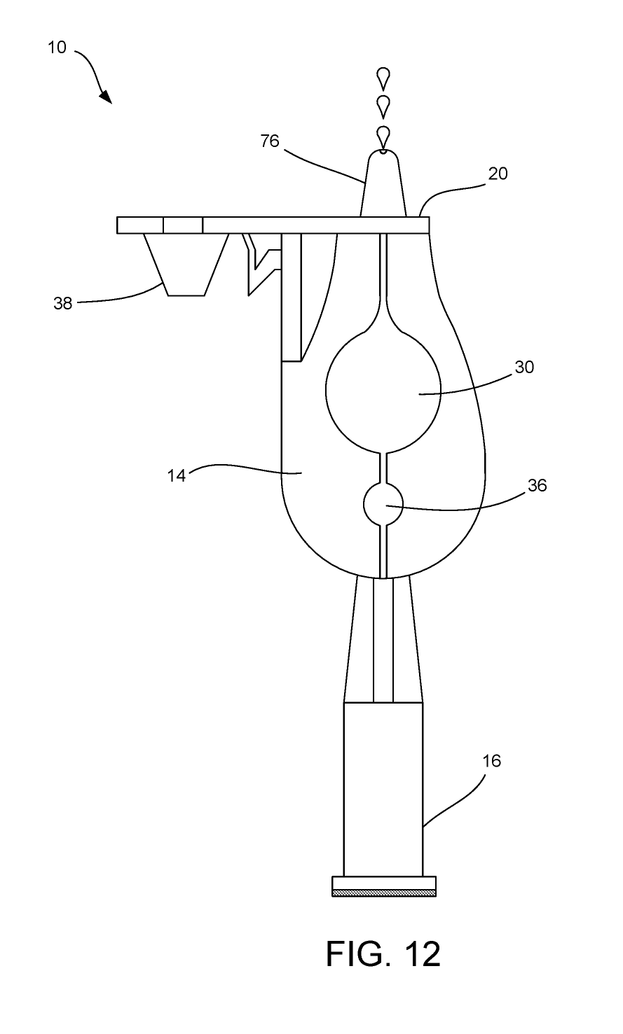

FIG. 12 is a side view of the single use delivery device 10 including an administration member 76 for delivering a fluid agent. The administration member 76 is generally in the form of a nozzle configured to facilitate droplet delivery of the fluid agent. FIG. 13 is a perspective view of the single use delivery device 10 including another embodiment of a droplet nozzle 78 for delivering a fluid agent. A delivery device 10 including the droplet nozzle 76, 78 may be useful for delivery of fluid agents in which the preferred route of administration is via droplet delivery, such as intraocular delivery (e.g., droplets in the eyes, oral administration, topical administration, and the like).

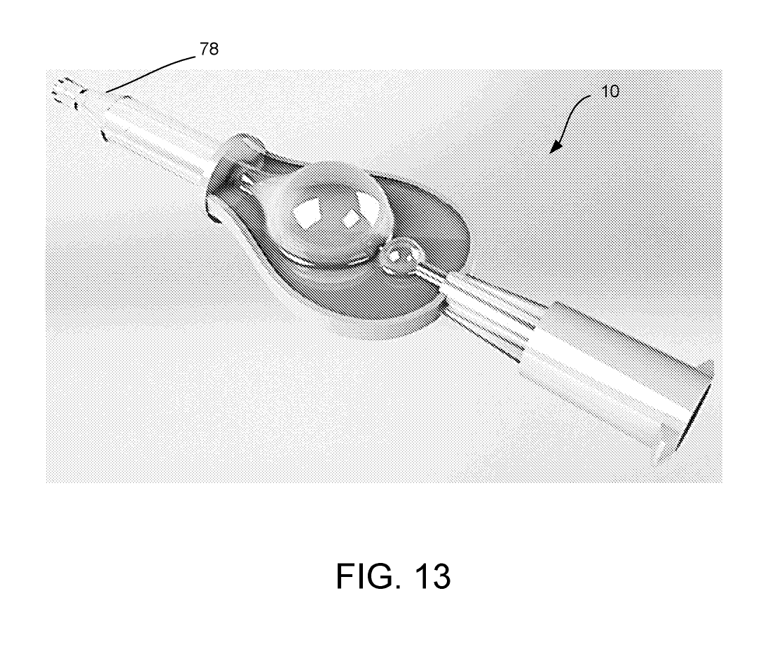

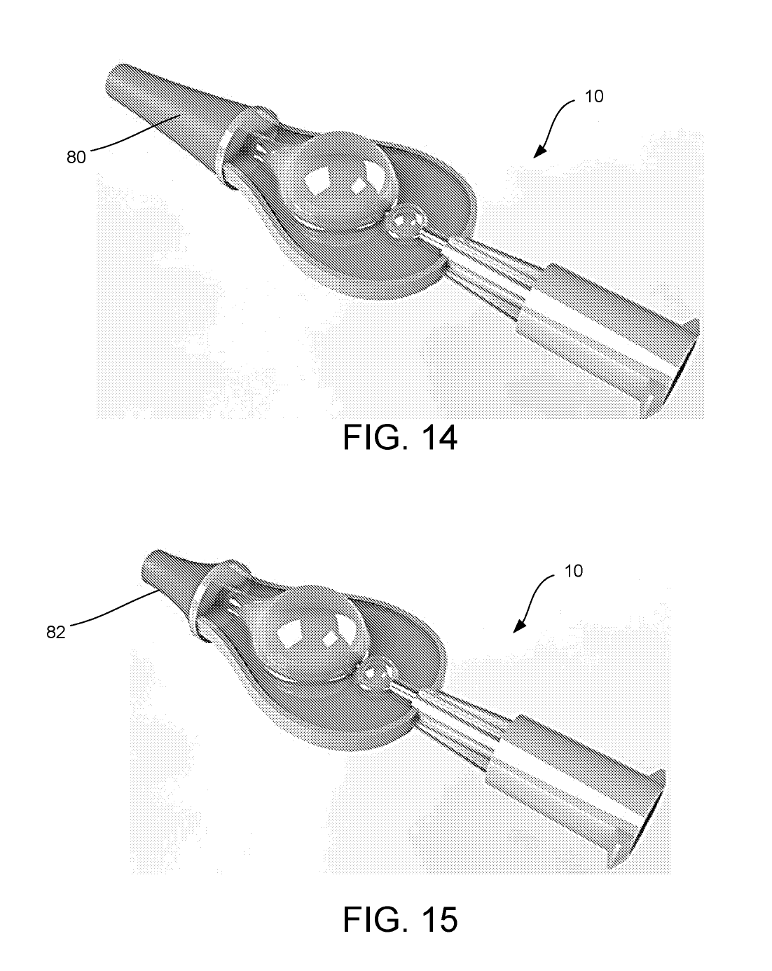

FIG. 14 is a perspective view of the single use delivery device 10 including another embodiment of an administration member 80 for delivering a fluid agent within a patient's ear canal. FIG. 15 is a perspective view of the single use delivery device 10 including another embodiment of an administration member 82 for delivering a fluid agent, particularly useful for topical application to a wound or other site.

The delivery device is configured to allow delivery of the agent to the patient in a relatively simple manner, without requiring specialized training for injecting a needle portion intradermally. In particular, the delivery device is designed such that it may be filled on-site and in the field with a microdose of an agent, while remaining sterile and preventing the potential for contamination during the filling process. For example, when filling the delivery device with a fluid agent, a person need only couple a filler syringe containing the fluid agent to the inlet port and then fill the reservoir with the fluid agent by applying pressure to a plunger of the filler syringe. Due to the one-way valve, the fluid agent is only permitted to flow within the reservoir and prevented from flowing in a retrograde fashion out of the reservoir. Furthermore, the interior volume of the reservoir may be within a range considered to be a micro dose. Thus, the delivery device does not require exact measurements when filling the reservoir. Instead, a person need only completely fill the reservoir, which includes the correct dosage, and further prevents overfilling, as the interior volume is limited to the dosage amount for any given fluid agent.

Because the delivery device itself is not prefilled, the delivery device of the present invention does not require the maintenance of a certain temperature (e.g., 2 to 8 degrees Celsius) during shipment or storage, thus cutting down on the overall costs. Rather than maintaining the delivery device at a constant temperature, as is the case with current devices, only the source containing the vaccine or drug (e.g., single supply provided in filling syringe) need by maintained at a constant temperature. Additionally, because the delivery device is configured to store and deliver a microdose of agent, the delivery device allows for dose-sparing. Dose-sparing may provide for a successful immunization program, particularly in resource-poor settings, by potentially reducing the per-injection cost (including transport and storage) of vaccines because more doses might be obtained from the existing vaccine presentation. Dose-sparing might also extend the availability of vaccines in cases where supply is limited by manufacturing capacity. Accordingly, a plurality of empty delivery devices may be shipped and stored, at a reduced cost, and then filled directly on-site and on an as-needed basis, such that only a single filler syringe is required for hundreds of doses to be delivered at any given point.

Once filled, the delivery device is designed such that a person administering the agent (e.g., administrator) need only press the delivery device against the administration site (e.g., shoulder, arm, chest, etc.), in which the device is configured such that needle penetration is limited to the correct length and orientation within the administration site. For example, in some embodiments, the needle is positioned substantially perpendicular relative to a plane along which the distal end of the base member lies, such that the needle is configured to be inserted into a patient's skin at a substantially perpendicular angle and the distal end is configured to contact the patient's skin indicating adequate depth of penetrating for intradermal injection of the fluid agent.

Upon needle penetration, the administrator then may fully compress a reservoir containing the micro dose of agent, thereby delivering the correct predefined dosage to the patient. The delivery device is further configured to be rendered incapable of reuse following its delivery of the agent to a patient, thereby preventing reuse of the device and reducing the risk of the spreading blood-borne diseases through reuse. For example, in some embodiments, the reservoir member is configured to substantially collapse and reduce the interior volume upon substantial compression applied thereto. In particular, the top member may include an inelastic material such that the reservoir member is prevented from being reformed and the interior volume prevented from expanding subsequent to substantial compression. In some embodiments, the top member may further include a valve cover configured to substantially enclose the one-way valve within. Upon substantial compression applied to the valve cover, the valve cover is configured to substantially collapse upon the one-way valve and render the one-way valve inoperable, thereby blocking fluid flow from the inlet port to the reservoir member.

Furthermore, the delivery device may be configured to prevent unintentional needle sticks, and thus reduce the potential for spreading blood-borne diseases. For example, in some embodiments, the base member further includes a needle protector member extending from distal end adjacent to the outlet port. The needle protector member is configured to move between a closed position, in which a penetrating tip of the needle is shielded, and an open position, in which the penetrating tip of the needle is exposed.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A single use delivery device comprising:
an administration member for administering a fluid agent into a patient;
a base member for providing said fluid agent into said administration member, said base member comprising:
a proximal end having an inlet port configured to receive said fluid agent from a source and a distal end having an outlet port coupled to said administration member and configured to provide said fluid agent thereto;
a channel providing a fluid pathway from said inlet port to said outlet port; and
a one-way valve positioned within said fluid pathway of said channel, said one-way valve configured to limit fluid flow to an antegrade direction from said inlet port toward said outlet port; and
a top member coupled to said base member and comprising a compressible reservoir member in fluid communication with said fluid pathway of said channel, said reservoir member having an interior volume configured to receive and store said fluid agent passing through said one-way valve and configured to expel said fluid agent into said fluid pathway and through said outlet port into said administration member in response to a compression force applied thereto.

2. The single use delivery device of claim 1, wherein said administration member comprises a needle for at least one of subcutaneous, intramuscular, intradermal, and intravenous injection of said fluid agent into said patient.

3. The single use delivery device of claim 2, wherein said needle is a micro-needle having a length in the range of 0.5 mm to 4 mm.

4. The single use delivery device of claim 2, wherein said needle has a length in the range of 4 mm to 15 mm.

5. The single use delivery device of claim 2, wherein said needle has a length in the range of 15 mm to 30 mm.

6. The single use delivery device of claim 1, wherein said administration member comprises a nozzle configured to control administration of said fluid agent to said patient.

7. The single use delivery device of claim 6, wherein said nozzle is configured to facilitate dispersion of said fluid agent into a spray.

8. The single use delivery device of claim 6, wherein said nozzle is configured to facilitate dispersion of said fluid agent into one or more droplets.

9. The single use delivery device of claim 1, wherein said reservoir member is configured to substantially collapse and reduce said interior volume upon substantial compression applied thereto.

10. The single use delivery device of claim 9, wherein said top member comprises an inelastic material such that said reservoir member is prevented from being reformed and said interior volume prevented from expanding subsequent to substantial compression.

11. The single use delivery device of claim 1, wherein said base member further comprises a protector member extending from said distal end adjacent to said outlet port and configured to move between a closed position, in which a tip of said administrator member is shielded, and an open position, in which said tip of said administration member is exposed.

12. The single use delivery device of claim 11, wherein said protector member is coupled to said distal end via a living hinge.

13. The single use delivery device of claim 1, wherein said top member further comprises a valve cover configured to substantially enclose said one-way valve.

14. The single use delivery device of claim 13, wherein, upon substantial compression applied to said valve cover, said valve cover is configured to substantially collapse upon said one-way valve and render said one-way valve inoperable, thereby blocking fluid flow from said inlet port to said reservoir member.

15. The single use delivery device of claim 1, wherein an interior volume of said reservoir member is in the range of 0.05 ml to 1.0 ml.

16. The single use delivery device of claim 1, wherein each of said base member and said top member comprises a medical grade material.

17. The single use delivery device of claim 1, wherein said top member comprises a thermoplastic polymer.
